(19) United States
(12) Patent Application Publication
(10) Pub. No.: US 2018/0014717 A1
(43) Pub. Date: Jan. 18, 2018

(54) Adaptor for Multiple Types of Endoscope

(71) Applicant: Cook Medical Technologies LLC, Bloomington, IN (US)

(72) Inventors: Niall Benn, Co. Limerick (IE); Aaron Collins, Co. Limerick (IE); Michael S. Clancy, Limerick (IE)

(73) Assignee: Cook Medical Technologies LLC, Bloomington, IN (US)

(21) Appl. No.: 15/647,796

(22) Filed: Jul. 12, 2017

Related U.S. Application Data
(60) Provisional application No. 62/361,816, filed on Jul. 13, 2016.

(51) Int. Cl. A61B 1/00

(52) U.S. Cl. CPC A61B 1/00121; A61B 1/00114; A61B 1/00066; A61B 1/00193

(57) ABSTRACT

An adaptor and method for connecting a medical device to an endoscope are provided where the adaptor and the endoscope have non-compatible connectors. The adaptor includes a first portion including a proximal end portion having a mating connector for connecting to the elongate medical device and a distal end adapted to operably connect to an accessory channel of the endoscope. The first portion also includes a flange positioned distal to the mating connector and having a diameter greater than a diameter of the mating connector and a tubular body extending distally from the flange to the distal end, the tubular body being sized and shaped for insertion at least partially into the accessory channel. A lumen of the first portion extends through the first portion from the proximal end portion to the distal end where the lumen is adapted to receive a portion of the medical device therethrough.

### RELATED APPLICATIONS

This application claims the benefit of U.S. Provisional Application No. 62/361,816, filed Jul. 13, 2016, which is incorporated by reference herein in its entirety.

### TECHNICAL FIELD

This invention generally relates to adaptors for receiving elongate medical devices that are insertable into an endoscope, and in particular to adaptors for connecting to an accessory channel port of multiple types of endoscope.

### BACKGROUND

Endoscopic devices and procedures may be used to diagnose, monitor and treat various conditions by close examination of the internal organs. By way of background, a conventional endoscope generally is an instrument having a device for visualizing the interior of an internal region of a body and a lumen for inserting one or more treatment devices therethrough. A wide range of applications have been developed for the general field of endoscopes including by way of example the following: arthroscope, angioscope, bronchoscope, choledochoscope, colonoscope, cytoscope, duodenoscope, enteroscope, esophagogastro-duodenoscope (gastroscope), laparoscope, laryngoscope, nasopharyngo-neproscope, sigmoidoscope, thoracoscope, and utererscope (individually and collectively, “endoscope”).

In some endoscopic devices, visualization of the internal regions may be obtained using a video camera. The video camera provides a viewing field to observe the surgical instrumentation or procedure within the viewing field. Medical ultrasound has also been used to monitor a surgical procedure within a viewing field. Endoscopic ultrasound (EUS) utilizes high frequency sound waves to create an image of living tissue or an echogenic surface. Ultrasound waves are emitted from transducers located at the distal end of an endoscope. Surgical instruments having an echogenic surface reflect the ultrasound waves and enable an endoscopist to monitor the location of the device within the patient.

In some procedures, medical devices are inserted through the endoscope to access the internal organs. For example, an elongate device, such as a needle, may be inserted through an accessory channel of the endoscope for diagnostic and therapeutic procedures.

Endoscopes manufactured by different companies may have different types of connectors at the accessory channel port for connecting with elongate medical devices. The different type of connector at the accessory channel port requires that each elongate medical device include the appropriate connector for each different type of endoscope. The requirement for different connectors on a variety of different types of elongate medical devices greatly increases the need for increased inventory of the medical devices, depending on the type of endoscope that will be used during a medical procedure. The increased inventory also increases the costs associated with these medical procedures.

It is desirable to have an adaptor that is universally connectable to the accessory channel port of any type of endoscope, and which includes a portion that is sized and shaped to receive an elongate medical device chosen by the endoscopist. It is also desirable to a have an adaptor that may be used with multiple types of endoscope that this securable to each type of endoscope and free from rotation relative to the endoscope when secured thereto.

### SUMMARY OF THE INVENTION

Accordingly, it is an object of the present invention to provide an adaptor for an endoscope having features that resolve or improve on one or more of the above-described drawbacks.

The foregoing object is obtained in one aspect by providing an adaptor configured to connect a medical device to an endoscope, the adaptor and the endoscope having non-compatible connectors. The adaptor includes a first portion. The first portion includes a proximal end portion having a mating connector for connecting to the elongate medical device and a distal end adapted to operably connect to an accessory channel of the endoscope. The first portion also includes a flange positioned distal to the mating connector and having a diameter greater than a diameter of the mating connector and a tubular body extending distally from the flange to the distal end, the tubular body being sized and shaped for insertion at least partially into the accessory channel. A lumen of the first portion extends through the first portion from the proximal end portion to the distal end where the lumen is adapted to receive a portion of the medical device therethrough.

Methods of securing a medical device to an endoscope where the medical device and the endoscope have incompatible connectors are provided. Methods include inserting a tubular body of a first portion of an adaptor into an endoscope so that a distal end of the first portion extends into an accessory channel of the endoscope and operably contacting the first portion with a connector of the endoscope so that a mating connector on a proximal end portion of the first portion extends proximal to the connector of the endoscope so that the medical device is connectable to the mating connector on the first portion.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF THE DRAWINGS

FIG. 1 is a partial side perspective view of an endoscope having an accessory channel with a connector;

FIG. 2 is partial a side perspective view of an endoscope having an accessory channel with an alternative connector to the connector shown in FIG. 1;

FIG. 3 is a partial sectional view of an adaptor in accordance with an embodiment of the present invention with the adaptor connected to the endoscope;

FIG. 4 is a sectional view of the second portion of the adaptor shown FIG. 3;

FIG. 5 is a partial side view of the adaptor shown in FIG. 3;

FIG. 6 is a partial side view of the adaptor of FIG. 3;

FIG. 7 is a partial side view of an embodiment of an adaptor having a locking portion;

FIG. 8 is a partial side view of the adaptor shown in FIG. 7 with the locking portion in a closed position;

FIG. 9 is a side perspective view of an embodiment of an adaptor in accordance with the present invention;

FIG. 10 is a sectional view of an embodiment of the adaptor shown in FIG. 9;

FIG. 11 is an exploded view of an embodiment of an adaptor in accordance with the present invention;

FIG. 12 is a perspective view of the adaptor shown in FIG. 11;

FIG. 13A is a perspective view of the adaptor shown in FIG. 12;

FIG. 13B is a partial perspective view of the adapter shown in FIG. 13A being inserted into an endoscope;

FIG. 14A is a perspective view of the adaptor shown in FIG. 9;

FIG. 14B is a partial perspective view of the adapter shown in FIG. 14A inserted into an endoscope;

FIG. 15 is a perspective view of an adaptor according to the present invention;

FIG. 16 is a perspective view of the adaptor shown in FIG. 15 closely fit to an accessory channel; and

FIG. 17 is a perspective view of the adaptor shown in FIG. 15 with the adaptor closed and connected to the accessory channel.

### DETAILED DESCRIPTION OF THE EMBODIMENTS

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

As used in the specification, the terms proximal and distal should be understood as being in the terms of a physician operating an endoscope and an elongate medical device for insertion into a patient. Hence the term distal means the portion of the device that is farthest from the physician and the term proximal means the portion of the device that is nearest to the physician.

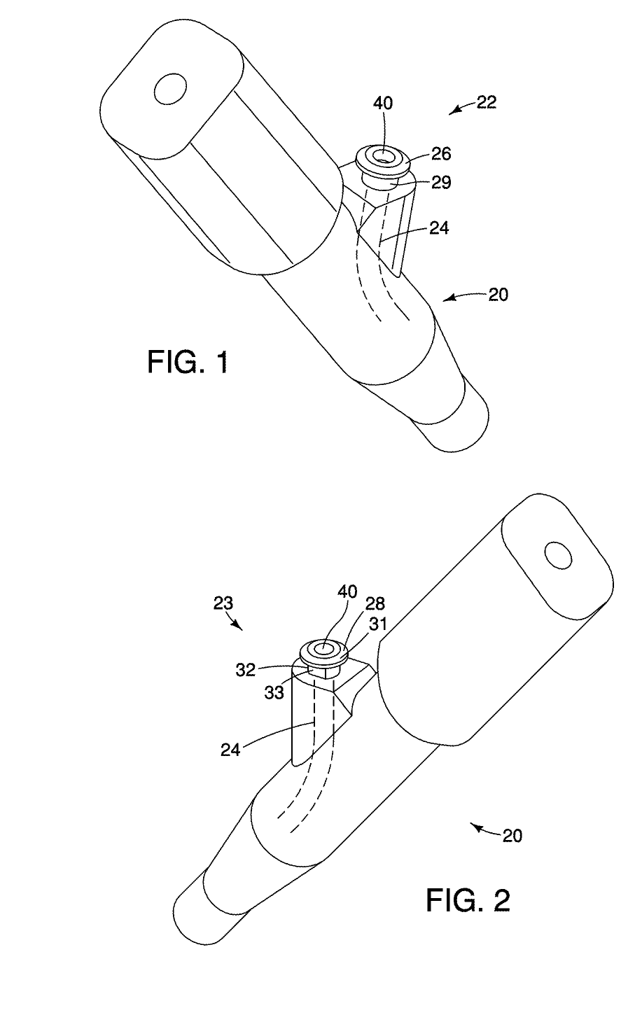

FIGS. 1 and 2 illustrate an exemplary portion of an endoscope 20 showing two different types of connectors 22, 23 that may be used for connecting a medical device to an accessory channel 24 of the endoscope 20. Other types and sizes of connectors on the endoscope 20 are also possible. FIG. 1 illustrates a portion of the endoscope 20 having a connector 22 for connecting a medical device to the accessory channel 24 of the endoscope 20. FIG. 1 illustrates the connector 22 having a flange 26 and a stem 29 with a cylindrical shape for connection with the medical device and a lumen 40 extending through the connector 22. FIG. 2 illustrates a different type of a connector 23 for connecting the medical device to the endoscope 20 that requires a different connection portion on the medical device than the connecting portion on the medical device for connecting to the connector 22 shown in FIG. 1. The connector 23 shown in FIG. 2 includes a flange 31 and a stem 32 having two flat sides 33 on opposite sides of the stem 32 for connection with the medical device and a lumen 40 extending through the connector 23.

The embodiments of the adaptors described herein may be used with any type of connector on an endoscope for connecting a medical device to the connector so that the adaptor allows for universal connection of the medical device to different types connectors on the accessory channel of the endoscope. The adaptors described herein will reference connection to exemplary connector 22, however, one skilled in the art will understand that the adaptors are configured to connect to any type of connector on the accessory channel of the endoscope.

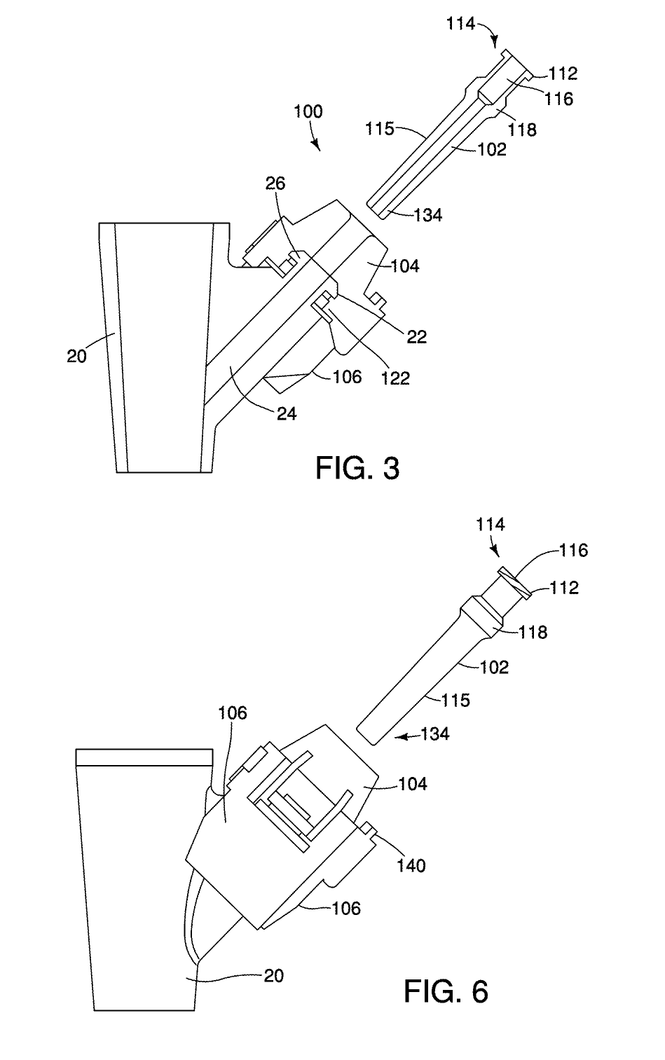

FIG. 3 illustrates a sectional view of an embodiment of an adaptor 100 for connecting medical devices to connector 22 of the accessory channel 24 of the endoscope 20 in accordance with embodiments of the present invention. The adaptor 100 includes a first portion 102 that includes a mating insert and a second portion 104 that is adapted to receive the mating insert portion 102 and to connect the adaptor 100 to the connector 22 on the endoscope 20. As shown in FIG. 3, the mating insert portion 102 includes a mating connector such as a luer-type connector 112 on a proximal portion 114 of the first portion 102 that is configured to mate with a portion of the medical device. The first insert portion 102 also includes a tubular body 115 having a lumen 116 extending therethrough that is sized and shaped to receive a portion of the medical device that is to be inserted into the working channel 24 of the endoscope 20. In some embodiments described herein, the tubular body 115 may taper inward toward a distal end 134 of the tubular body 115. The first portion 102 may also include a flange 118 on the proximal portion 114 and positioned distal to the mating connector 112. The flange 118 may be positioned at a distance away from the mating connector 112 to correctly position the medical device for connection to the endoscope 20. In some embodiments, the flange 118 may be adjacent to the mating connector 112. The flange 118 has a diameter that is greater than a diameter of the mating connector. The flange 118 may be gripped by the operator to inset the first portion 102 into the second portion 104 as described below.

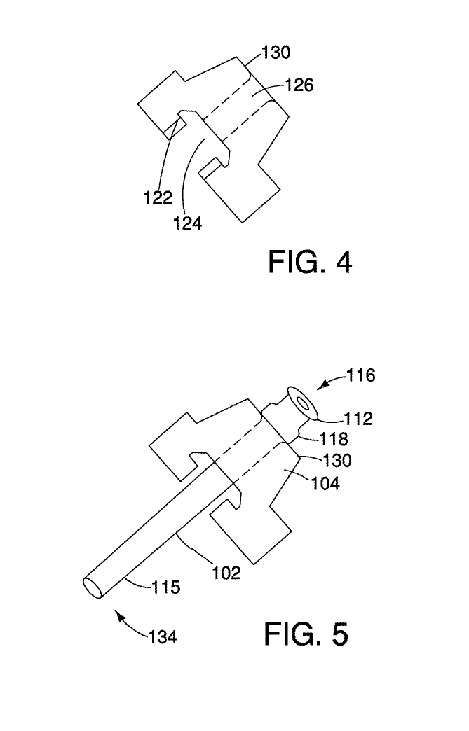

The second portion 104 of the adaptor 100 may be shaped as a cap that is sized and shaped to fit over the connector 22 and to surround the flange 26 of the connector 22. In some embodiments the second portion 104 may be generally cylindrical or may be conically shaped. The second portion 104 may include a flange 122 that extends beneath the flange 26 of the connector 22 to hold the second portion 104 on the connector 22. The second portion may include an opening 124 sized and shaped to fit over the connector 22 of the endoscope 20 so that a portion of the second portion abuts the flange 26 of the connector 22. The opening 124 and the flange 122 of the second portion 104 are sized so that the opening 124 accepts any connector on the endoscope 20, for example when the stem has a cylindrical configuration or the stem includes one or more flat portions. The flange 122 is also adapted to fit beneath the flange of any connector. See FIGS. 3 and 4 illustrating the second portion 104 and the opening 124.

The second portion 104 includes a lumen 126 extending therethrough that operably connects with the accessory channel 24 of the endoscope 20. As shown in FIG. 5, the tubular body 115 of the first portion 102 may be inserted through the lumen 126 of the second portion 104 until the flange 118 contacts an upper surface 130 of the second portion 104. The lumen 126 of the second portion 104 may be sized and shaped so that the tubular body 115 of the first portion 102 fits securely within the lumen 126, such as by friction fit. The first portion 102 has a length that extends through the lumen 126 of the second portion 104 and the distal end 134 of the first portion 102 extends into the working channel 24 of the endoscope 20. The second portion 104 may be made from a flexible, elastomeric material so that the second portion 104 can be flexed to fit over the connector 22. The adaptor 100 is configured for removable connection to the endoscope 20. The first portion 102 and the second portion 104 may be independently movable in relation to each other and may be assembled together and then connected to the connector 22 or may be separately connected to the connector 22 by connecting the second portion 104 to the connector 22 and inserting the first portion 102 through the lumen 126 of the second portion 104.

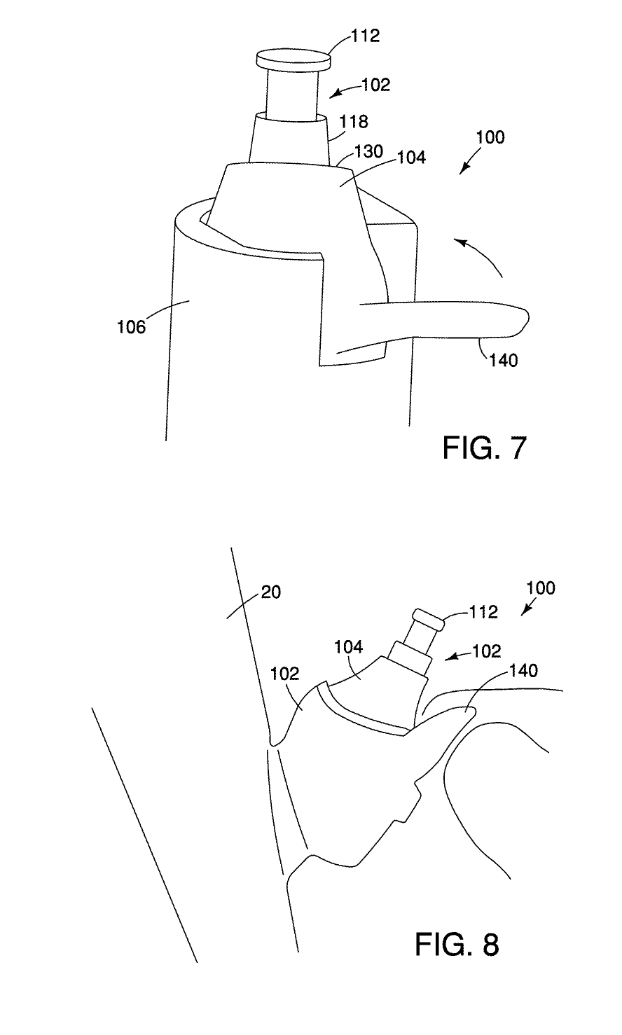

In some embodiments, the adaptor 100 may include a third portion 106 that fits over the second portion 104 and secures the second portion 104 to the connector 22 of the endoscope 20. FIGS. 6-8 illustrate the third portion 106 which is sized and shaped to fit over the second portion 104. In some embodiments, the third portion 106 may be made of a material that is harder than the second portion such as a rigid plastic third portion 106 fit over a rubber or elastomeric second portion 104. The third portion 106 is shown positioned over the second portion 104 in FIG. 6 with the first portion 102 to be inserted into the lumen 126 of the second portion 104. FIGS. 7 and 8 illustrate the tubular body 115 of the first portion 102 inserted into the lumen 126 of the second portion 104 so that the flange 118 of the first portion abuts the upper surface 130 of the second portion 104. A portion of the second portion 104 may extend proximally out of the third portion 106 so that the first portion 102 may be readily inserted in to the lumen 126 and into the accessory channel 24. In some embodiments, the third portion 106 may circumferentially surround the second portion 104 around the flange 122 to facilitate positioning of the flange 122 below the flange 26 of the connector 22.

In some embodiments, the third portion 106 may include a locking tab 140 that is movable from an open position shown in FIG. 7 toward a closed position shown in FIG. 8. With the locking tab 140 in the closed position, the adaptor 100 is secured to the connector 22 of the endoscope 20 as shown in FIG. 8. The locking tab 140 may be moved to the open position to remove the adaptor 100 from connection to the endoscope 20. In some embodiments, the locking tab 140 is moved to the closed position after the first portion 102 is inserted into the second portion 104. In some embodiments, the third portion 106 may be preassembled with the second portion 104 so that the operator connects the second portion 104 with the third portion 106 to the connector 22. The first portion 102 may be inserted into the lumen 126 before or after connection of the second portion 102 to the connector 22.

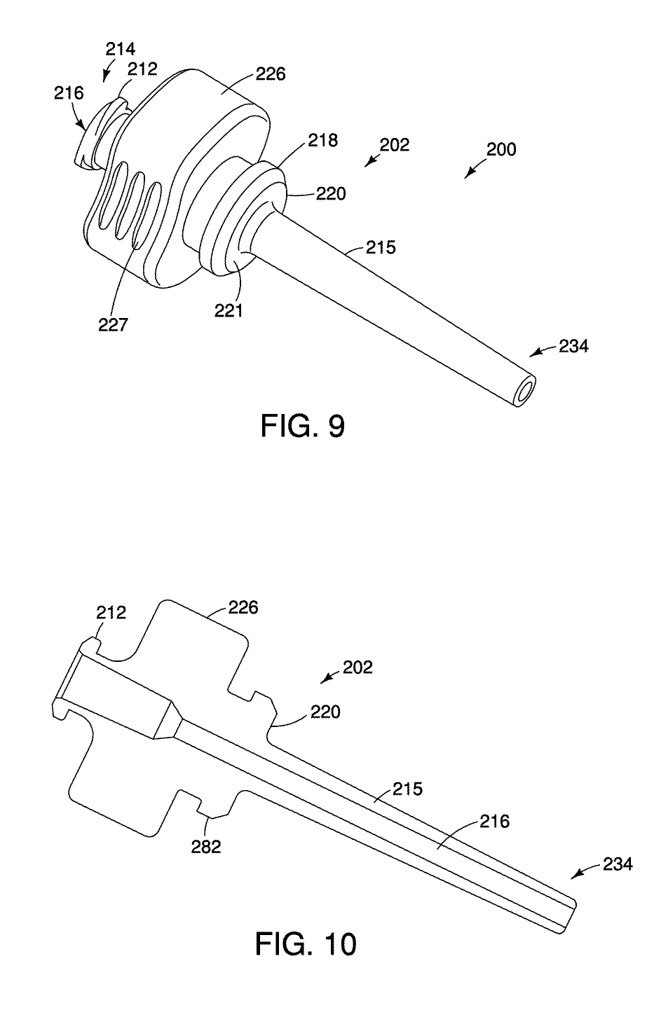

FIGS. 9 and 10 illustrate an embodiment of an adaptor 200 for connecting medical devices to the connector 22 of the accessory channel 24 of the endoscope 20 in accordance with embodiments of the present invention. The adaptor 200 includes a first mating insert portion 202. A tubular body 215 of the mating insert portion 202 is sized and shaped to insert into the accessory channel 24 of the endoscope 20 so that a distal portion 234 of the first insert portion 202 is positionable in the accessory channel 24 when the first portion 202 is operably connected to the endoscope 20. The first insert portion 202 includes a mating connector such as a luer-type connector 212 on a proximal portion 214 of the first portion 202. A lumen 216 extends through the first portion 202 and connects to the accessory channel 24 so that the medical device can connect to the mating connector 212 and extend through the tubular body 215 and into the accessory channel 24 of the endoscope 20 via the lumen 216 of the first portion 202. The first insert portion 202 also includes a flange 218 that has a lower surface 220 that is adapted to contact the connector 22 of the endoscope 20. The flange 218 has a diameter that is greater than a diameter of the luer-type connector 212. In some embodiments, the lower surface 220 may include a groove 221 that is adapted to mate with an upper surface of the connector 22. In some embodiments, the lower surface 220 may be flat.

In some embodiments, the first portion 202 may include a gripping flange 226 that has one or more indentations 227 to receive a thumb or finger of the operator to facilitate insertion or removal of the adaptor 200 into/out of the accessory channel 24. The gripping flange 226 may be positioned between the mating connector 212 and the flange 218 and the gripping flange 226 may have a larger diameter that the diameters of the mating connector 212 and the flange 218. In some embodiments, the adaptor 200 is made of unitary construction so that the adaptor 200 is a single piece insert. The single piece insert adaptor 200 may be inserted into the endoscope 20 so that the tubular body 215 is at least partially positioned within the accessory channel 24 and the lower surface of the flange 218 operably contacts the flange 26 of the connector 22 of the endoscope 20. As described below and shown in FIG. 14B, the adaptor 200 may also be used with a flexible seal that fits over the connector 22.

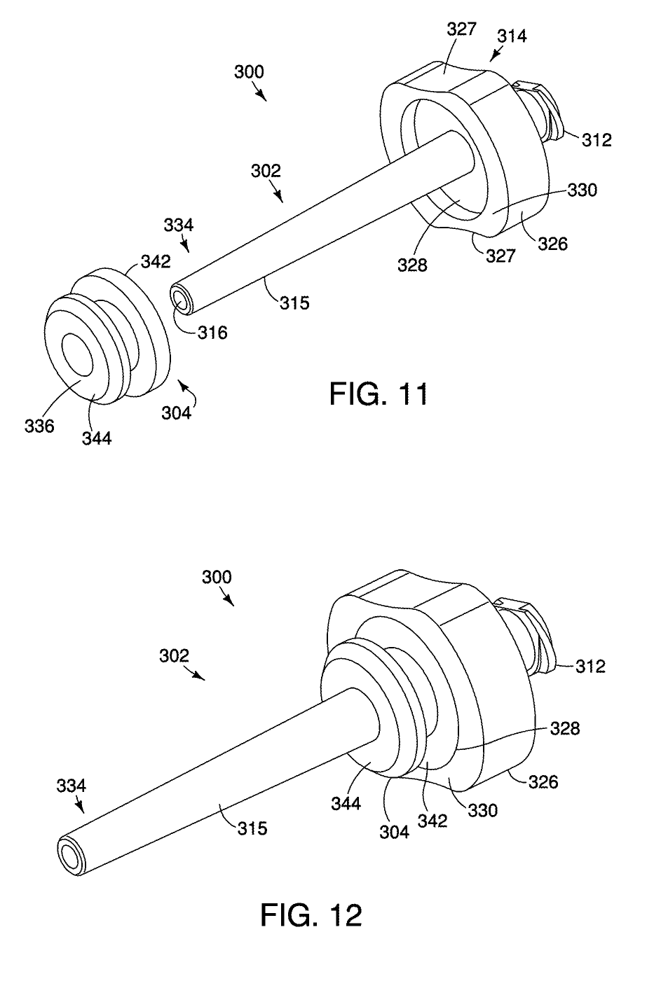

FIGS. 11 and 12 illustrate an embodiment of an adaptor 300 for connecting medical devices to the connector 22 of the accessory channel 24 of the endoscope 20 in accordance with embodiments of the present invention. The adaptor 300 includes a first mating insert portion 302 and a second portion 304. The second portion 304 is shown separate from the first portion 302 in FIG. 11 and connected to the first portion 302 in FIG. 12. A tubular body 315 of the first mating insert portion 302 is sized and shaped to insert into the accessory channel 24 of the endoscope 20 so that a distal portion 334 of the first insert portion 302 is positionable in the accessory channel 24 when the first portion 302 is operably connected to the endoscope 20. The first insert portion 302 includes a mating connector such as a luer-type connector 312 on a proximal portion 314 of the first portion 302. A lumen 316 extends through the first portion 302 and connects to the accessory channel 24 so that the medical device can connect to the mating connector 312 and extend through the tubular body 315 and into the accessory channel 24 of the endoscope 20 via the lumen 316 of the first portion 302.

The first insert portion 302 may include a gripping flange 326 that may have one or more indentations 327 to receive a thumb and/or finger of the operator to facilitate insertion or removal of the adaptor 300 into/out of the accessory channel 24. A diameter of the gripping flange 326 is greater than a diameter of the mating connector 315. The gripping flange 326 may also include a groove 328 on a lower surface 330 of the gripping flange 326 that is sized and shaped to receive a portion of the second portion 304 in the groove 328. In some embodiments, the groove 328 may be circular to receive a circularly shaped second portion 304. In other embodiments, the groove may be oval or curvilinear or any shape that can receive a portion of the second portion 304. The shape of the groove 328 can be configured to mate with a similarly shaped proximal end 342 of the second portion 304.

The second portion 304 includes a lumen 336 extending therethrough that is sized and shaped to receive the tubular body 315 of the first portion 302. FIG. 12 illustrates the tubular body 315 extending through the lumen 336 of the second portion 304 and the proximal end 342 of the second portion 304 positioned in the groove 328 of the first portion 302. The second portion 304 includes a distal surface 344 that is adapted to contact the connector 22 of the endoscope 20. The lower surface 344 may be flat or may include a groove that is adapted to mate with an upper surface of the connector 22 similar to the embodiments described above. The second portion 304 may be made from a flexible material that is positioned against the connector 22 of the endoscope 20 so the second portion 304 can be positioned against any size or shaped connector 22 when the adaptor 300 is inserted into the accessory channel 24 of the endoscope 20.

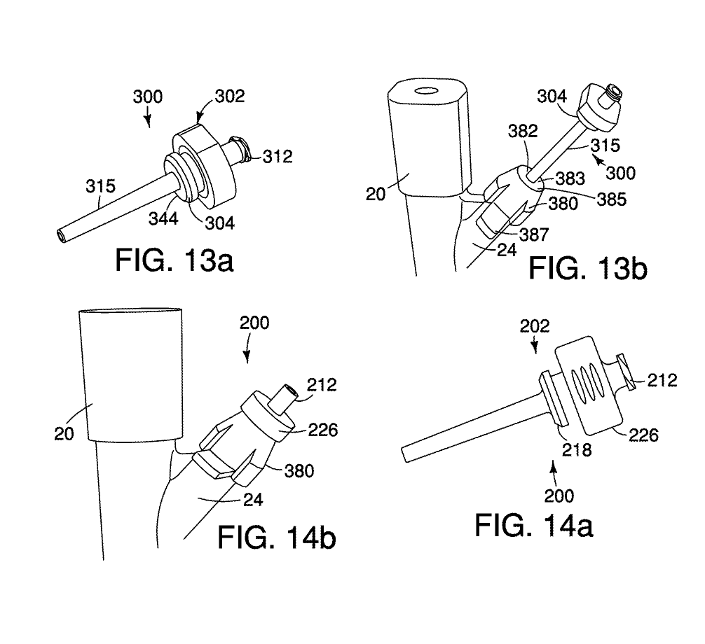

In some embodiments, the adaptor 200 and the adaptor 300 may be used together with a flexible seal 380 as shown in FIGS. 13B and 14B. The flexible seal 380 fits over the existing connector 22 and the body of the endoscope 20 at the accessory channel 24. The seal 380 includes an opening 382 for a lumen 383 that operably connects to the accessory channel 24. The seal 380 may include one or more tabs 384 to help facilitate placement of the seal 380 over the connector 22 of the endoscope 20. The lumen 383 of the seal 380 is sized to receive the tubular body 215, 315 of the first portion 202, 302, respectively. In some embodiments, the second portion 304 may be positioned within the opening 383. Alternatively, the lower surface 344 of the second portion 304 may abut an upper surface 385 of the adaptor 380. In both alternatives, the tubular body 315 extends through the lumen 383 and into the accessory channel 24 of the endoscope 24.

FIGS. 14A and 14B illustrate the adaptor 200 being used with the seal 380. As shown in FIG. 14B, the adaptor 200 may be inserted through the opening 382 through the lumen 383 of the seal 380 so that the tubular body 215 is positioned within the accessory channel 24 of the endoscope 20. The flange 218 may be positioned within the opening 382 so that the gripping flange 226 is adjacent to the upper surface 385 of the seal 380.

When the adaptor 200, 300 is positioned with the tubular body 215, 315 in the accessory channel 24, the mating connector 212, 312 is positioned so that the medical device may be connected to the mating connector 212, 312 and extend through tubular body 215, 315 and into the accessory channel 24 of the endoscope 20.

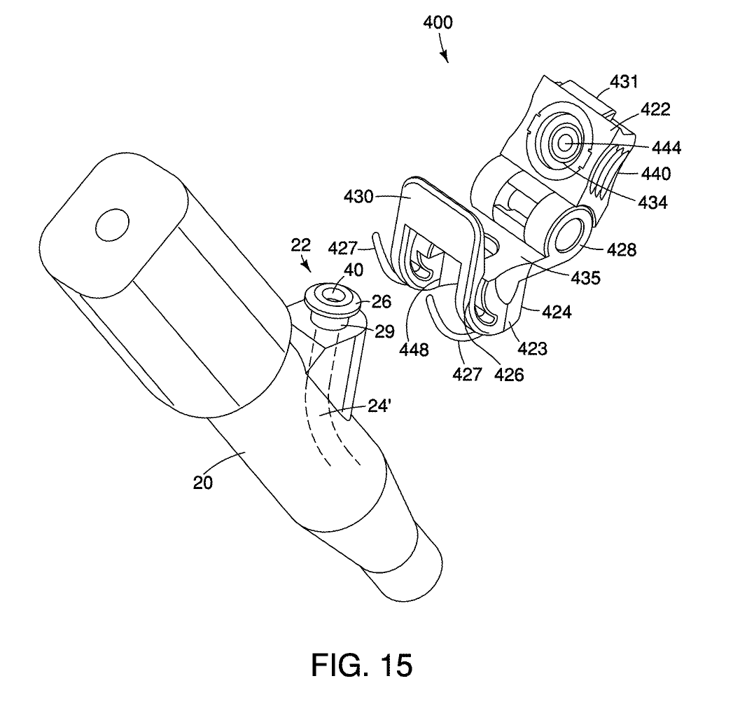

FIGS. 15-17 illustrate an embodiment of an adaptor 400 of the present invention. An adaptor 400 is shown in FIG. 15 having a first portion 422 and a second portion 424. As shown in FIG. 15, the first portion 422 may include a recess 432 that is sized and shaped to receive the connector 22 and form a seal against the connector 22. In some embodiments, a separate sealing member 434 may be provided to allow the elongate medical device to pass through the sealing member 434 and prohibit fluid and contamination from entering or exiting the adaptor 400. The first portion 422 includes an opening 444 defined through the first portion 422 having the seal 434 therein and operably connecting to the accessory channel 24 of the endoscope 20. A mating connector 450 may be provided at the opening 444 for simple connection of an elongate medical device to the adaptor 400. Any type of connector 450 that connects with the desired elongate medical device may be provided on the first portion 422 of the adaptor 400. The elongate medical device can be connected to the connector 450 of the adaptor 400 so that the shaft of the elongate medical device extends through the openings 444 and 40 and into the accessory channel 24. The first portion 422 may include one or more gripping portions 440 to help facilitate opening and closing of the first portion 422 relative to the second portion 424.

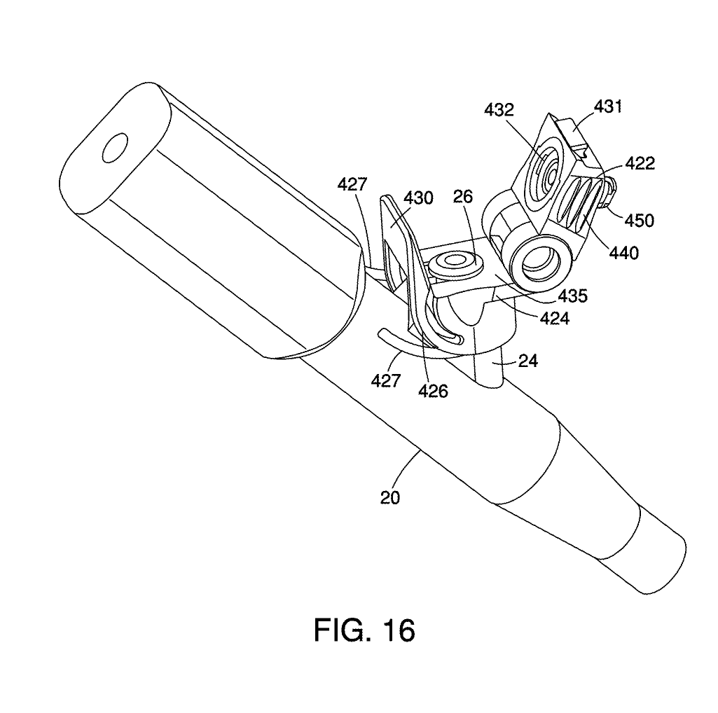

The second portion 424 is configured to complimentarily engage the endoscope 20 and fit around a stem portion 29 and beneath a flange 26 of the connector 22 connected to the accessory channel 24. The second portion 424 includes an opening 448 extending through the second portion 424 and defined by a body portion 423 that is sized and shaped to fit closely with the accessory channel 24 of the endoscope 20. The second portion 424 is configured to laterally slide over the accessory channel portion of the endoscope 20 so that a surface 435 of the second portion 424 slides beneath the flange 26 of the connector 22. In some endoscopes 20, as described above, the connector 22 may be provided with flat side portions 31on the stem portion 29. Depending on the endoscope 20, the flat side portions may be in different orientations. The opening 448 in the second portion 424 of the adaptor 410 allows the body portion 423 of the second portion 424 to hug the connector 22 and fit over the stem portion 29 of the connector 22, regardless of the orientation of the flat side portions or the size of a cylindrical or oval stem. The second portion 424 further includes the surface 435 that is sized and shaped to fit beneath the flange 26 of the connector 22 to hold the adaptor 400 against the endoscope 20. The connector 22 extends through the opening 448 and the flange 26 extends over a portion of the surface 435 when the second portion 424 is closely fit onto the endoscope around the accessory channel 24 as shown in FIG. 16. The second portion 424 may also include a closure 430 for closing and securing the first portion 422 as described below. The closure 430 may be connected to the body portion 423 by a pair of flexible legs 426. The second portion 424 may also include a second pair of flexible legs 427 that are sized and shaped to fit over the endoscope 20 to rotationally secure the adaptor 400 on the endoscope 20.

The second portion 424 of the adaptor 400 may be connected to the first portion 422 by a hinge 428 as shown in FIGS. 15 and 16. Alternatively, the second portion 424 may be provided separately from the first portion 422 or connected by any means known to one skilled in the art. The adaptor 400 may be provided with the hinge 428 in an open position so that the second portion 424 is laterally slidable over the portion 29 of the connector 22 and beneath the flange 26 and then the first portion 422 may be secured over the connector 22 and to the second portion 424 by the closure 430 connected to a corresponding closure 431 on the first portion 422. The flange 26 is secured between the first portion 422 and the second portion 424 of the adaptor 400. In some embodiments, the closure 430 is formed integrally with the first portion 422 and snaps over the closure 431 of the second portion 424 to secure the adaptor 410 to the connector 28 of the accessory channel 30. Alternatively, the closure 430 may be provided as a separate piece that secures the first portion 422 together with the second portion 424 and onto the connector 28. The second portion 424 may also include gripping members 440 to facilitate closure of the second portion onto the first portion.

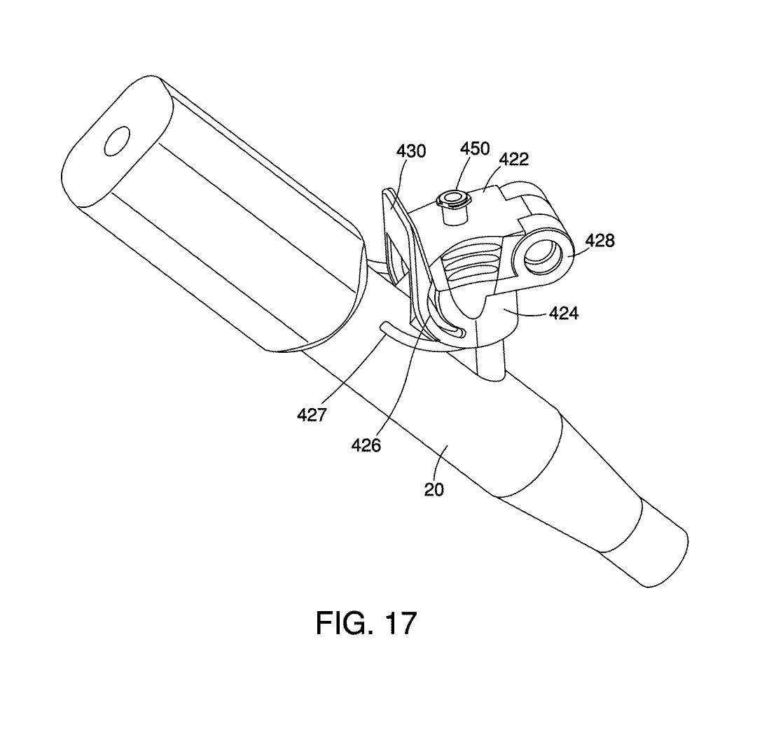

FIG. 17 illustrates the adaptor 400 secured to the portion 29 of the connector and the first portion 424 secured to the second portion 422 and the flange 26 therebetween. The second set of flexible arms 427 are secured around the endoscope 20 to rotationally secure the adaptor 400. An elongate medical device may be connected to the connector 450 and the device inserted through the opening 444. The second set of flexible arms 427 are positioned on opposite sides of the endoscope 20 so that the adaptor 400 remains in position when the medical device is connected and disconnected to the adaptor 400 and when the medical device is in use through the accessory channel 24.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims. Application of the principles of the invention to any other elongate medical device are within the ordinary skill in the art and are intended to be encompassed within the scope of the attached claims.

1. An adaptor for connecting a medical device to an endoscope, the medical device and the endoscope having non-compatible connectors, the adaptor comprising:
a first portion comprising:
a proximal end portion having a mating connector for connecting to the elongate medical device,
a distal end adapted to operably connect to an accessory channel of the endoscope;
a first flange distal to the mating connector and having a diameter greater than a diameter of the mating connector;
a tubular body extending distally from the first flange to the distal end, the tubular body being sized and shaped for insertion at least partially into the accessory channel; and
a lumen extending through the first portion from the proximal end portion to the distal end, the lumen adapted to receive a portion of the medical device therethrough.

2. The adaptor of claim 1, further comprising a second portion having a surface for contacting the connector of the endoscope and a lumen extending therethrough, the lumen of the second portion being sized and shaped to receive the tubular body therethrough.

3. The adaptor of claim 1, wherein the first flange comprises a gripping portion comprising one or more indentations.

4. The adaptor of claim 1, further comprising a second flange, the second flange comprising a gripping portion.

5. The adaptor of claim 2, wherein the second portion comprises a flange adapted to extend beneath the connector on the endoscope.

6. The adaptor of claim 2, wherein the second portion has a greater flexibility than the first portion.

7. The adaptor of claim 2, wherein the second portion surrounds the connector of the endoscope and the tubular body of the first portion extends through the second portion and into the accessory channel.

8. The adaptor of claim 2, wherein the second portion at least partially fits in a groove of on a lower surface of the first portion so that the second portion is positioned between the connector of the endoscope and the first portion.

9. The adaptor of claim 2, further comprising a third portion wherein the third portion at least partially fits over the second portion and the third portion secures the second portion over the connector.

10. The adaptor of claim 9, wherein the third portion is rigid relative to the second portion.

11. The adaptor of claim 9, wherein the third portion comprises a locking tab movable from an open position to a closed position to secure the adaptor to the endoscope.

12. The adaptor of claim 4, wherein the first flange is positionable within the lumen of the second portion and the distal end of the tubular body extends into the accessory channel.

13. The adaptor of claim 1, further comprising a seal that fits over the connector of the endoscope and the first portion extends through a lumen of the seal so that the distal end of the tubular body extends into the accessory channel.

14. A method of securing a medical device to an endoscope where the medical device and the endoscope have incompatible connectors, the method comprising:
inserting a tubular body of a first portion of an adaptor into an endoscope so that a distal end of the first portion extends into an accessory channel of the endoscope; and
operably contacting the first portion with a connector of the endoscope so that a mating connector on a proximal end portion of the first portion extends proximal to the connector of the endoscope so that the medical device is connectable to the mating connector on the first portion.

15. The method of claim 14, comprising positioning a flexible second portion over the connector of the endoscope and inserting the tubular body of the first portion through a lumen of the second portion.

16. The method of claim 15, comprising positioning a third portion over a portion of the second portion to secure the second portion to the connector of the endoscope.

17. The method of claim 16, comprising moving a locking tab on the third portion from an open position to a closed position to secure the second portion to the connector of the endoscope.

18. The method of claim 16, comprising inserting the tubular body through the lumen of the second portion before positioning the third portion of the second portion.

19. The method of claim 14, comprising positioning a seal over the connector of the endoscope and inserting the tubular body of the first portion though a lumen of the seal so that a distal end of the tubular body is positioned in the accessory channel.

20. The method of claim 14, further comprising inserting a medical device into the adaptor so that a distal end of the medical device extends through the tubular body and in into the accessory channel and connecting the medical device to the mating connector of the first portion.
